(19) United States
(12) Patent Application Publication
(10) Pub. No.: US 2013/0035526 A1
(43) Pub. Date: Feb. 7, 2013

(54) PROCESS FOR THE MANUFACTURE OF TETRAFLUOROOLEFINS

(71) Applicants: Maher Y. Elsheikh, Wayne, PA (US); Philippe Bonnet, Lyon (FR); Benjamin Bin Chen, Wayne, PA (US)

(72) Inventors: Maher Y. Elsheikh, Wayne, PA (US); Philippe Bonnet, Lyon (FR); Benjamin Bin Chen, Wayne, PA (US)

(73) Assignee: Arkema Inc., King of Prussia, PA (US)

(21) Appl. No.: 13/640,326

(22) PCT Filed: Apr. 8, 2011
(86) PCT No.: PCT/US11/31689

§ 371 Date: Oct. 10, 2012

Related U.S. Application Data
(60) Provisional application No. 61/324,068, filed on Apr. 14, 2010.
(60) Provisional application No. 61/413,650, filed on Nov. 15, 2010.

(51) Int. Cl. C07C 17/25; C07C 17/20

(52) U.S. Cl. 570/156; 570/155; 570/170

(57) ABSTRACT

A method for producing a tetrafluoroolefin, such as 2,3,3,3-tetrafluoropropene (HFO-1234yf), comprises contacting 1,1,1,2-tetrachloro-2-fluoropropane (HCFC-241bb) with or without a catalyst under conditions effective to convert the 1,1,1,2-tetrachloro-2-fluoropropane (HCFC-241bb) to the tetrafluoroolefin, optionally, via an intermediate. The conversion may be a one-step fluorination or a two-step fluorination and dehydrochlorination process. The 1,1,1,2-tetrachloro-2-fluoropropane (HCFC-241bb) may also be obtained by dehydrochlorinating 1,2,3-trichloropropane (HCC-260da) to form 2,3-dichloropropene (HCO-1250xf); fluorinating 2,3-dichloropropene (HCO-1250xf) to form 1,2-dichloro-2-fluoropropane (HCFC-261bb); and chlorinating 1,2-dichloro-2-fluoropropane (HCFC-261bb) to form 1,1,1,2-tetrachloro-2-fluoropropane (HCFC-241bb).

### FIELD OF THE INVENTION

The invention relates to a method of making tetrafluoroolefins, such as 2,3,3,3-tetrafluoropropene (HFO-1234yf), from different feedstocks and intermediates.

### BACKGROUND OF THE INVENTION

Chlorine-containing compounds such as chlorofluorocarbons (CFCs) are considered to be detrimental to the Earth's ozone layer. Many of the hydrofluorocarbons (HFCs) used to replace CFCs have been found to contribute to global warming. Therefore, compounds that do not damage the environment, but also possess the properties necessary to function as refrigerants, solvents, cleaning agents, foam blowing agents, aerosol propellants, heat transfer media, dielectrics, fire extinguishing agents, sterilants and power cycle working fluids, have been investigated. Fluorinated olefins, especially those containing one or more hydrogens in the molecule (referred to herein as hydrofluoroolefins (HFOs)) are being considered for use in some of these applications, such as in refrigeration as well as in processes to make fluoropolymers. In particular, HFO-1234yf may be useful as a refrigerant composition and has a lower potential to contribute to global warming compared to refrigerant compositions, such as HFC-134a.

The manufacture of tetrafluoroolefins, such as HFO-1234yf, has been shown to suffer from a number of drawbacks, such as custom manufactured catalysts, expensive manufacturing costs, multiple-step processes, high pressure hydrogen fluoride (HF) activation, etc. In particular, multistep processes are generally more complicated and less economical compared to shorter synthesis routes. For example, the multiple step fluorination of 241bb to 1234yf may include a catalytic or non-catalytic dehydrochlorination of 241bb to 1231yf (step 1), isomerization of the 1231yf to the olefin 1231ya (step 2), and gas phase fluorination of the 1231ya to 1234yf (step 3). Accordingly, there remains a need for more direct routes and better catalyst selection to convert readily available and inexpensive starting materials.

### SUMMARY OF THE INVENTION

The methods according to the present invention provide practical industrial methods for manufacturing tetrafluoroolefins, and particularly, HFO-1234yf. The methods of the present invention and the catalysts selected are believed to provide reactions with high conversion and good selectivity.

According to an embodiment of the present invention, a method for producing a tetrafluoroolefin comprises contacting 1,1,1,2-tetrachloro-2-fluoropropane (HCFC-241bb) with or without a catalyst under conditions effective to convert the 1,1,1,2-tetrachloro-2-fluoropropane (HCFC-241bb) to the tetrafluoroolefin, optionally, via an intermediate, such as 1,1,1,2-tetrafluoro-2-chloropropane (HCFC-244bb). The conversion may be a one-step fluorination process or a two-step process, first fluorination followed by dehydrochlorination. The fluorination may be a gas phase or liquid phase fluorination, which may depend upon the starting materials selected.

According to another embodiment of the present invention, a method for producing 2,3,3,3-tetrafluoropropene (HFO-1234yf) comprises converting 1,1,1,2-tetrachloro-2-fluoropropane (HCFC-241bb) to 2,3,3,3-tetrafluoropropene (HFO-1234yf). The converting step may be performed in a gas phase or a liquid phase. For example, the converting step may be a one-step process comprising fluorinating 1,1,1,2-tetrachloro-2-fluoropropane (HCFC-241bb) to form 2,3,3,3-tetrafluoropropene (HFO-1234yf).

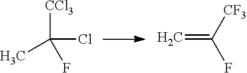

For example, if the one-step process is a gas phase fluorination, the fluorination may occur in the presence of a chromium containing catalyst. If the one-step process is a liquid phase fluorination, the fluorination may occur in the presence of a superacid, such as an antimony halide.

Alternatively, the converting step may be a two-step process comprising fluorinating 1,1,1,2-tetrachloro-2-fluoropropane (HCFC-241bb) to form 1,1,1,2-tetrafluoro-2-chloropropane (HCFC-244bb) and dehydrochlorinating 1,1,1,2-tetrafluoro-2-chloropropane (HCFC-244bb) to form 2,3,3,3-tetrafluoropropene (HFO-1234yf).

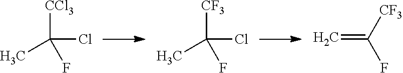

According to another embodiment of the present invention, a method for producing 2,3,3,3-tetrafluoropropene (HFO-1234yf) comprises dehydrochlorinating 1,2,3 trichloropropane (HCC-260da) to form 2,3-dichloropropene (HCO-1250xf); fluorinating 2,3-dichloropropene (HCO-1250xf) to form 1,2-dichloro-2-fluoropropane (HCFC-261bb); chlorinating 1,2-dichloro-2-fluoropropane (HCFC-261bb) to form 1,1,1,2-tetrachloro-2-fluoropropane (HCFC-241bb); and converting 1,1,1,2-tetrachloro-2-fluoropropane (HCFC-241bb) to 2,3,3,3-tetrafluoropropene (HFO-1234yf) by either the one-step or two-step process.

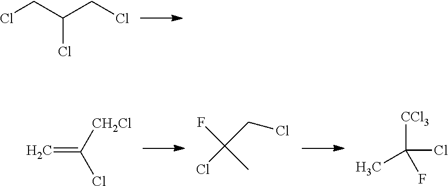

According to another embodiment of the present invention, a method of forming an intermediate for use in producing a tetrafluoroolefin comprises fluorinating 1,1,1,2-tetrachloro-2-fluoropropane (HCFC-241bb) to form 1,1,1,2-tetrafluoro-2-chloropropane (HCFC-244bb). The tetrafluoroolefin may be produced by contacting the intermediate 1,1,1,2-tetrafluoro-2-chloropropane (HCFC-244bb) with a catalyst, such as chlorine gas or anhydrous nickel salt, under conditions effective to convert the 1,1,1,2-tetrafluoro-2-chloropropane (HCFC-244bb) to the tetrafluoroolefin.

### BRIEF DESCRIPTION OF THE DRAWING

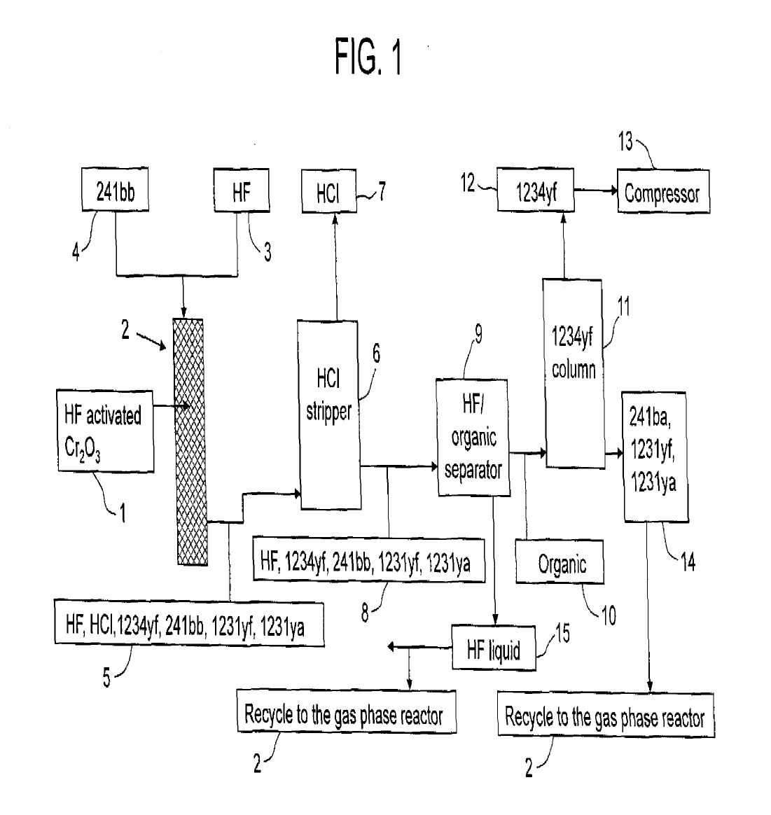

The invention may be further understood by reference to a drawing in which FIG. 1 depicts a flowchart of a gas phase fluorination process that may be used to manufacture 1234yf using 241bb as a feedstock.

### DETAILED DESCRIPTION OF THE INVENTION

Aspects of the present invention include methods for producing tetrafluoroolefins, such as 2,3,3,3-tetrafluoropropene (HFO-1234yf), from feedstocks directly and/or indirectly by obtaining preferred intermediates.

According to one embodiment of the present invention, a method for producing a tetrafluoroolefin comprises contacting 1,1,1,2-tetrachloro-2-fluoropropane (HCFC-241bb) with or without a catalyst under conditions effective to convert the 1,1,1,2-tetrachloro-2-fluoropropane (HCFC-241bb) to the tetrafluoroolefin, optionally, via an intermediate, such as 1,1,1,2-tetrafluoro-2-chloropropane (HCFC-244bb).

As used herein, HFO designates hydrofluoroolefins, HCO designates hydrochloroolefins, HFC designates hydrofluorocarbons, and HCFC designates hydrochlorofluorocarbon. Each species may be discussed interchangeably with respect to its chemical formula, chemical name, abbreviated common name, etc. For example, 2,3,3,3-tetrafluoropropene may be designated as CH₂═CFCF₃, HFO-1234yf, or 1234yf. Also, some compounds may be described with respect to their ASHRAE (American Society of Heating, Refrigerating and Air-Conditioning Engineers) designations, such as R-241bb for 1,1,1,2-tetrachloro-2-fluoropropane. Table 1 provides a representative list.

TABLE 1

| | | | |
|---|---|---|---|
| 2,3,3,3-tetrafluoropropene | CH₂═CFCF₃ | HFO- | 1234yf |
| | | 1234yf | |
| 1,1,1,2-tetrachloro-2- | CH₃CFClCCl₃ | HCFC- | 241bb |
| fluoropropane | | 241bb | |
| 1,1,1,2-tetrafluoro-2- | CH₃CFClCF₃ | HCFC- | 244bb |
| chloropropane (also known | | 244bb | |
| as 2-chloro-1,1,1,2- | | | |
| tetrafluoropropane) | | | |
| 1,2,3-trichloropropane | CH₂ClCHClCH₂Cl | HCC- | 260da |
| | | 260da | |
| 2,3-dichloropropene | CH₂═CCl(CH₂Cl) | HCO- | 1250xf |
| | | 1250xf | |
| 1,2-dichloro-2-fluoropropane | CH₃CFClCH₂Cl | HCFC- | 261bb |
| | | 261bb | |

Each compound described herein, unless designated otherwise, includes its different isomers and stereoisomers, including all single configurational isomers, single stereoisomers, and any combination thereof in any ratio.

A tetrafluoroolefin is the ultimate reaction product desired although it is envisioned that other reaction products and intermediates may also be produced using the methods described herein. In an exemplary embodiment, the tetrafluoroolefin is a tetrafluoropropene. For example, the tetrafluoropropene may be obtained directly from a tetrachlorofluoropropane or from an intermediate compound, such as a chloro-tetrafluoropropane. Preferably, the tetrafluoropropene is 2,3,3,3-tetrafluoropropene or HFO-1234yf, which is a fluorinated hydrocarbon with the formula CH₂═CFCF₃. HFO-1234yf is a non-ozone-depleting fluorocarbon replacement with a low global warming potential, which has been under development as a refrigerant. In particular, HFO-1234yf may be suitable as a refrigerant for mobile air conditioning (MAC) applications.

In an exemplary embodiment of the present invention, a method for producing 2,3,3,3-tetrafluoropropene (HFO-1234yf) comprises converting 1,1,1,2-tetrachloro-2-fluoropropane (HCFC-241bb) to 2,3,3,3-tetrafluoropropene (HFO-1234yf). As used herein, the term “converting” includes direct converting (e.g., a single reaction or under essentially one set of reaction conditions) and indirect converting (e.g., two or more reactions or using more than a single set of reaction conditions).

It has been found that the HFO-1234yf may be efficiently produced by several different single and multiple step conversions. In an exemplary embodiment, HFO-1234yf may be obtained directly or indirectly from 1,1,1,2-tetrachloro-2-fluoropropane (HCFC-241bb). Without wishing to be bound to a particular theory, it is believed that the large difference in boiling points between the desired, 1234yf product and the 241bb feedstock and possible intermediates, such as 1231ya (CCl₂═CHCH₂Cl) and 1231yf (CH₂═CFCCl₃) may facilitate recovery of the 1234yf product.

### One-Step Conversion: Fluorinating

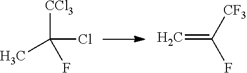

In a single step conversion, at least one tetrachlorofluoropropane, e.g., 1,1,1,2-tetrachloro-2-fluoropropane (HCFC-241bb) is directly converted to a tetrafluoroolefin, such as HCO-1234yf. The reaction may be catalytic or non-catalytic. The reaction may be conducted in liquid phase, vapor phase, or a combination of gas and liquid phases.

HCFC-241bb may be obtained or formed from any suitable source. For example, the starting material, 241bb, may be prepared according to A. Henne et al., J. Am. Chem. Soc, 1941, 63, 2692 incorporated herein by reference in its entirety for all purposes.

The direct conversion is preferably a fluorination process. The fluorination reaction introduces fluorine into the compound and chlorine is removed from the compound to form the tetrafluoroolefin. In other words, a source of fluorine is contacted with the tetrachlorofluoropropane during the reaction. Any suitable source of fluorine, such as hydrogen fluoride (HF), may be used. In an exemplary embodiment, hydrogen fluoride is the source of fluorine used during the fluorination step. The source of fluorine may be gaseous or of any other suitable type appropriate for the reaction. The fluorination conditions may also be of any suitable type, such as gas or liquid phase.

The fluorination may occur in the presence or absence of a catalyst. If a catalyst is used, any suitable catalyst may be selected. It has been found that a chromium-based catalyst (e.g., chromium (III)) is particularly effective in gas phase fluorination. Alternatively, a catalyst composed of a superacid or Lewis acid catalyst comprising an element selected from Sb, Sn, Ti, Ta, Nb and B, and the like may be used for liquid phase fluorination.

Liquid Phase Fluorination

A liquid phase fluorination may be suitable to produce 1234yf in a single step, for example, when the starting/feed material contains 241bb (which is a solid at room temperature and atmospheric pressure). The reaction scheme may be summarized as follows in Scheme 1:

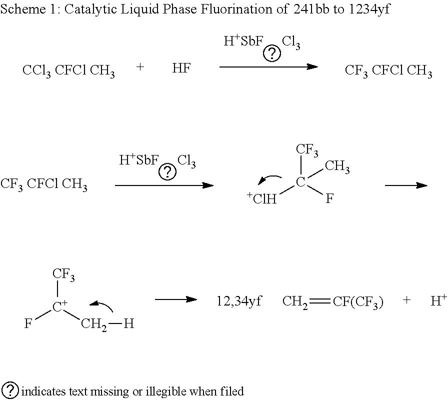

In particular, the liquid phase fluorination is more effective when starting with 241bb as the feed material because 241bb is a solid material at room temperature (i.e., standard conditions). Thus, gas phase fluorination of a solid 241bb material may be difficult to implement due to the nature of the feed and/or may require adjustments, e.g., by dissolving in an inert and stable solvent, such as perfluorohydrocarbon, or a polar solvent, such as liquid HF. Alternatively, 241bb can be fed as a melt into the gas phase reactor, for example.

The liquid phase fluorination may occur under any suitable conditions effective to convert the 241bb into the tetrafluoroolefin, 1234yf. For example, the fluorination may occur in the presence or absence of a catalyst. The liquid phase fluorination may occur in the presence or absence of a solvent. The process may be suitably carried out using batch or continuous conditions, which would be well known to those skilled in the art.

In one embodiment, the 1,1,1,2-tetrachloro-2-fluoropropane (HCFC-241bb) is converted into the tetrafluoroolefin using a one-step process comprising fluorinating the 1,1,1,2-tetrachloro-2-fluoropropane (HCFC-241bb) in the presence of a superacid catalyst to form the tetrafluoroolefin. When the process occurs in the liquid phase, it is preferred to use a catalyst comprising a superacid. A superacid is an acidic medium that has a proton-donating ability equal to or greater than 100% sulfuric acid (G. Olah et al.; SUPERACIDS, Wiley Intersciences, 1985, incorporated herein by reference). The superacid may be obtained from a Lewis acid. In particular, a homogenous, soluble, strong Lewis acid catalyst may be selected. In an exemplary embodiment, the Lewis acid comprises an element selected from Sb, Ti, Sn, B, Ta, Nb, and mixtures thereof, with halides (particularly chlorides and fluorides) of these elements being of particular interest. The Lewis acid may be formed into a superacid using any suitable means or techniques known in the art. Thus, the superacid may include an element selected from the group consisting of Ti, Sn, Nb, Ta, Sb, B, and mixtures thereof. In one embodiment, the selected Lewis acid halide is subjected to hydrogen fluoride (HF) activation in order to convert the Lewis acid halide into the corresponding fluoride or chlorofluoride salt. For example, the superacid may be of the form H⁺AClₓF_{y}⁻, where A is Ti, Sn, Nb, Ta, Sb, or B, Cl is chlorine, and F is fluorine. When A is Sb, Ta, or Nb, 0≦x≦6, and 0≦y≦6, and x+y=6. When A is Sn or Ti, x is 0≦x≦5, and 0≦y≦5, and x+y=5. When A is B, 0≦x≦4, and 0≦y≦4, and x+y=4. In an exemplary embodiment, the catalyst comprises antimony halide. It is envisioned, however, that any suitable acid or Lewis acid may be selected and used or converted into any suitable superacid effective to fluorinate the 241bb in the liquid phase.

Any suitable amount of catalyst may be used under any suitable conditions known in the art. For example, the level of catalyst used may be in the range between about 1-50 weight %, preferably between about 5-10 weight % of organic feed. The contact time for the liquid phase fluorination may vary between about 1-1000 minutes, which may depend on the strength and level of the catalyst used. For example, when more active catalysts are used, such as Sb, it is preferred to use shorter contact times and vice versa when less active catalysts, such as Sn or Ti, are used.

The feeds may be supplied at any suitable HF/241bb molar ratio. In a preferred embodiment, both HF and organic 241bb are fed at an approximate molar ratio of about 5-50 HF/241bb, preferably between about 10/1-20/1 molar ratio. Other suitable co-feeds may also be introduced to improve the reaction or maintain the catalyst activity for extended periods of time. For example, when the catalyst is a superacid or Lewis acid, Sb of variable oxidation states +3 and +5 may be especially desirable as an active catalyst. An antimony catalyst is an active catalyst if it is maintained in the higher oxidation state. On the other hand, the catalyst may lose its catalytic activity when reduced to the lower oxidation state. Therefore, it is beneficial to co-feed low levels of chlorine gas incrementally or continuously at a rate between 1-5 weight % to maintain the Sb catalyst active in the +5 oxidation state.

Gas Phase Fluorination

A gas phase fluorination may be suitable to produce a higher yield of 1234yf in a single step, via the intermediates 1231yf and the isomeric intermediate 1231ya, followed by allylic fluorination to form 1234yf Reaction scheme 2 may be summarized as follows:

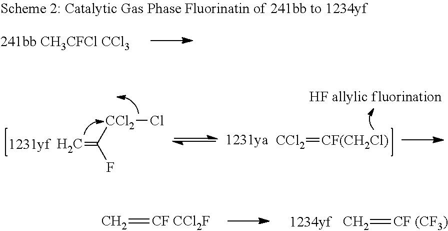

Any suitable catalyst may be selected when the process occurs in the gas phase. In one embodiment, it is preferred to use a chromium based solid catalyst, which may be supported or unsupported. Activated chromium (III) compounds, such as Cr₂O₃, are especially suitable. A suitable activated catalyst may be prepared as explained in U.S. Pat. No. 7,485,598, incorporated herein by reference for all purposes.

For example, the prepared chromium catalyst may be dried first using a temperature between about 100-200° C. in a stream of nitrogen for approximately 2-10 hours. Subsequently, the catalyst may undergo hydrogen fluoride (HF) activation at atmospheric or higher pressure (e.g., >150 lbs/square inch; PSI). If the catalyst was initially HF activated at atmospheric pressure, then it is preferably further HF activated under pressure in situ, prior to the start of feeding the organic. The operating temperature may be varied between about 100-500° C., preferably between about 200-400° C., and it is advantageous not to exceed 370° C. at any time during the course of activations. The resulting activated catalyst is preferably amorphous. The amorphous activated catalyst also preferably has the following characteristics: a minimum surface area of about 40 m²/g; pore volume (PV) greater than about 0.1 M³/g; catalyst attrition less than about 5%; crushing strength greater than about 40 PSI; and the weight % fluorine content about between 10-30 weight %, preferably 10-20 weight %. The surface catalytic active site is preferably equivalent to the CrOF compound and contains minimum amounts of the undesirable compound, CrF₃ (e.g., less than 1 weight % CrF₃).

The solid catalyst used for gas phase fluorination may be unsupported or supported. When supported, the catalyst may be supported using one or more suitable supports, such as activated carbon, graphite, chromia, alumina, zirconia, titania, magnesia, or the corresponding fluorinated compounds. In an exemplary embodiment, the catalyst comprises at least one support selected from the group consisting of alumina, fluorinated alumina, chromia, fluorinated chromia, activated carbon, and mixtures thereof. In a preferred embodiment, when chromium is the catalyst, the chromium is supported on HF pretreated activated carbon or alumina. When the catalyst is supported, the amount of catalyst carried thereon is suitably an effective amount, for example, about 0.1-80 total wt %, preferably about 1-20 total wt %, more preferably about 5-10 wt. % based on the total weight of the catalyst.

The catalyst may be used in the presence or absence of a co-catalyst. The catalyst does not require a co-catalyst, but a co-catalyst may be included therewith. For example, the chromium based solid catalyst may be combined with a co-catalyst, such as Ni, Zn, Co, Mn, Mg, and mixtures thereof. When present, the co-catalyst may be used at a low level, e.g., in the range of about 5-10 weight % based on the total weight of the catalyst. The co-catalyst may be added to the catalyst using any processes known in the art, such as mixed powder, co-precipitations or adsorption from aqueous or non-aqueous solutions. In an exemplary embodiment, the only catalytically active substance in the catalyst is chromium (i.e., the catalyst does not comprise a co-catalyst).

The physical shape of the catalyst is not particularly limited. In one embodiment, the catalyst is in the shape of pellets, powders, or granules. It is contemplated that the amount of catalyst used will vary depending on the particular parameters present during the reaction, which could be readily ascertainable by one of ordinary skill in the art.

The catalyst may be subjected to HF high temperature and/or high pressure activation. For example, the catalyst may be activated at a pressure of about 150 psig. In an exemplary embodiment, the catalyst is subjected to activation with HF. The activated catalyst may be of any suitable structure, e.g., amorphous or crystalline. In a preferred embodiment, the activated catalyst is amorphous with a surface area greater than 50 m²/g and a pore volume greater than 0.1 m³/g. The fluorine content present during HF activation may be of any suitable amount, but preferably is less than 22 weight %.

The conditions of the fluorination are not particularly limited. In one embodiment, the gas phase fluorination is carried out in the presence of a low level oxygen-containing gas, such as air, nitrogen, a nitrogen/oxygen mixture, etc. The oxygen level preferably is between about 0.01 to 1 volume % of organic feed (namely, the tetrachlorofluoropropane). As used herein, “organic” is intended to designate the primary reactant (i.e., 241bb) used in the reaction. The catalytic fluorination may also be carried out at any suitable temperature. In one embodiment, e.g., when 241bb is the tetrachlorofluoropropane, the gas phase fluorination is conducted at higher temperatures (e.g., about 200 to 400° C.).

After HF activation, the fluorination process may be carried out at a temperature between room temperature to 500° C., preferably about 100-500° C., more preferably about 200-400° C. A molar ratio of HF/organic may be in the range of about 1-50 HF/241bb, preferably within the range of about 10-20 HF/241bb. Any suitable contact time may be determined, such as a contact time between about 1-100 seconds, preferably about 1-60 seconds, more preferably about 10-30 seconds. The organic 241bb may be fed as a melt or preferably dissolved in inert perfluorinated solvent or polar solvent, such as liquid HF.

When the chromium based catalyst is used, it is preferred to use a low level of oxygen (e.g., fed as air at about 0.1-5 volume percent of organic feed) in order to maintain the catalyst activity for a longer period of time.

FIG. 1 depicts a flowchart of a gas phase fluorination process that may be used to manufacture 1234yf using 241bb as a feedstock. A high pressure activated Cr₂O₃ catalyst 1 is placed inside a gas phase reactor 2. The catalyst bed may be heated up in a stream of nitrogen at 200° C., for 4 hours. Subsequently, a mixture of HF 3 and organic 4 may be fed at a molar ratio of about 10/1 HF/244bb. In addition, a low level of oxygen, 2 volume %, may be added in the form of dry air as a co-feed to maintain the catalyst life for an extended period of time. The product 5 obtained includes HCl co-products and unreacted HF, organic products, such as 1234yf, 1231yf, 1231ya, and unreacted 241bb, which may be fractionated using HCl distillation column 6. The HCl co-product 7 may be collected at the top, and heavy organic 8, which may comprise 1234yf, 241bb, 1231yf, 1231ya, together with HF, may be admitted to HF separator 9. Liquid HF 15 may be collected at the bottom to be recycled back to the gas phase reactor 2. The light organic 10 may be fractionated using 1234yf light column 11. The desired organic product 1234yf 12 may be collected at the top and may be further sent to compressor 13. Meanwhile, heavy organic 14 together with un-reacted 241bb may be recycled back to the gas phase reactor 2.

Accordingly, in one embodiment, the 1,1,1,2-tetrachloro-2-fluoropropane (HCFC-241bb) is converted into the tetrafluoroolefin using a one-step process comprising fluorinating the 1,1,1,2-tetrachloro-2-fluoropropane (HCFC-241bb) in the presence of a chromium-containing catalyst to form the tetrafluoroolefin. In an exemplary embodiment, the converting step is a one-step process comprising fluorinating 1,1,1,2-tetrachloro-2-fluoropropane (HCFC-241bb) in the presence of a catalyst comprising chromium to form 2,3,3,3-tetrafluoropropene (HFO-1234yf). For example, a gas phase fluorination of 241bb may be conducted under a low level oxygen at high temperatures using a chromium-based catalyst. Without wishing to be bound to any specific reaction mechanism, it is believed that the fluorination process proceeds via β-elimination or γ-elimination. In either case, 1234yf results as the final product, possibly through a series of reactive intermediates believed to be halogenated cyclopropane compounds, as shown in Scheme 3.

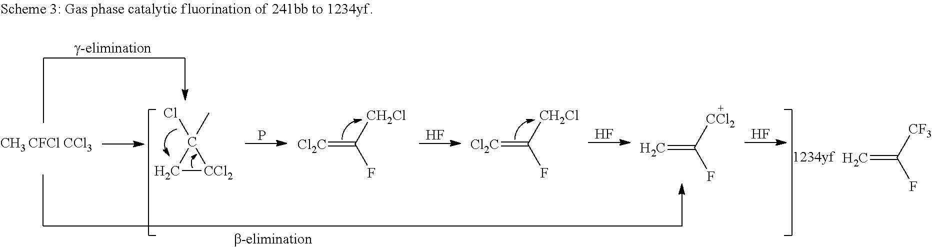

### Two-Step Conversion: Fluorinating and Dehydrochlorinating

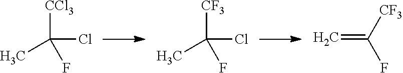

In a multiple-step conversion, multiple steps are required to produce the tetrafluoroolefin. For example, in a two-step conversion, a first step produces an intermediate, and in a second step, the intermediate is further reacted to produce the tetrafluoroolefin. In one embodiment of the present invention, the 1,1,1,2-tetrachloro-2-fluoropropane (HCFC-241bb) is converted into the tetrafluoroolefin using a two-step process comprising fluorinating the 1,1,1,2-tetrachloro-2-fluoropropane (HCFC-241bb) to form an intermediate; and subsequently, dehydrochlorinating the intermediate, in the presence or absence of a dehydrochlorination catalyst, to form the tetrafluoroolefin.

Step One: Fluorination

An intermediate suitable for use in producing the tetrafluoroolefin may be formed by fluorinating 1,1,1,2-tetrachloro-2-fluoropropane (HCFC-241bb) to form 1,1,1,2-tetrafluoro-2-chloropropane (HCFC-244bb). Thus, in an exemplary embodiment, the intermediate is 1,1,1,2-tetrafluoro-2-chloropropane (HCFC-244bb).

The discussion herein regarding the fluorination conditions and catalysts for the single step process applies equally here. For example, the reactions may be catalytic or non-catalytic, continuous or batch, conducted in liquid phase, vapor phase, or a combination thereof, etc. Thus, in an exemplary embodiment, a catalytic gas phase fluorination is used to convert the 1,1,1,2-tetrachloro-2-fluoropropane (241bb) to the 1,1,1,2-tetrafluoro-2-chloropropane (244bb) intermediate.

The feedstocks and intermediates shown in the prior art have some disadvantages. For example, some intermediates, such as 244bb, 245cb and/or 245eb, which may be formed from 1233xf (e.g., using 1230xa as a feedstock), may produce severe corrosion and form a high level of non-selective products. For example, a CF₃ group may favor product formation, such as 245eb, and the chlorine substituent may encourage the formation of 245eb and/or 245cb, as shown in Scheme 4.

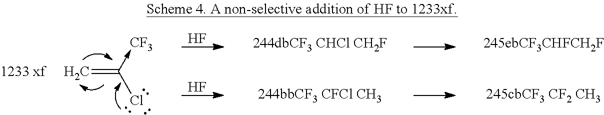

It has been found in the present invention, however, that it is advantageous to fluorinate 241bb to intermediate 244bb. In an exemplary embodiment, 241bb is fluorinated to 244bb in catalyzed liquid phase conditions. The fluorination preferably occurs in the presence of a catalyst. Any suitable catalyst may be selected. It has been found that a superacid or Lewis acid catalyst is particularly suitable. In an exemplary embodiment, the superacid or Lewis acid catalyst is selected from TiCl₄, SnCl₄, SbCl₅, TaCl₅, and the like.

The catalyst may be subjected to HF high temperature and/or high pressure activation. The catalyst may be activated using HF in gas or liquid phase. For example, the catalyst may be activated at a pressure of about 150 psig. In an exemplary embodiment, the catalyst is subjected to activation with HF. It is also recognized that any co-product gas, such as HCl, may be removed from the process as necessary.

The fluorination process may be conducted using any suitable conditions. The organic (e.g., 241bb) and HF may be fed to the reactor individually or as a mixture. For example, a mixture of HF and 241bb may be fed to the reactor at a molar ratio of HF/241bb between about 1/1-1000/1, preferably about 5/1 to 200/1, more preferably about 10/1-20/1. The contact time may, for example, be varied between about 1-100 minutes. The mixture of HF and 241bb may also contain the activated catalyst dissolved in a large excess of HF (e.g., 10-20 times the amount of 241bb). The reactor temperature may be between about 50 to 300° C., preferably between about 100-200° C. The reactor pressure may be about 100-1000 psig.

Step Two: Elimination

Once the intermediate is formed, step two includes converting the intermediate into the tetrafluoroolefin. Any suitable process of converting the intermediate may be used. For example, the reactions may be catalytic or non-catalytic, and the reactions may be conducted in liquid phase, vapor phase, or a combination thereof. In an exemplary embodiment, the second converting step is a dehydrochlorination/elimination reaction. Thus, a selective catalytic process of eliminating HCl from the 244bb intermediate may be used to manufacture 1234yf. Any suitable elimination catalyst may be used. In an exemplary embodiment, HCl elimination of 244bb occurs by using a radical initiator, e.g., chlorine gas or chlorine gas initiator, a transition metal-based catalyst, e.g., a nickel-based catalyst, as the dehydrochlorination catalyst, or some combination thereof.

With respect to the radical initiator, 1234yf may be produced by dehydrochlorination of 244bb using a free radical initiator as the dehydrochlorination catalyst. Irrespective of how 244bb is formed, one suitable method of dehydrochlorination may include contacting 1,1,1,2-tetrafluoro-2-chloropropane (HCFC-244bb) (or any molecule containing a hydrogen and chlorine on adjacent carbon atoms) with chlorine or a chlorine generator under free radical initiation conditions, which would be readily ascertainable by one of ordinary skill in the art, e.g., high temperature conditions.

In one embodiment, the intermediate is dehydrochlorinated in the presence of a chlorine gas free radical initiator as the dehydrochlorination catalyst. The chlorine gas may be introduced in any suitable way known in the art. For example, the chlorine or chlorine gas may be co-fed as pure or dilute chlorine gas, a chlorine generator or initiator (known to those skilled in the art, which may decompose, for example, to form chlorine), such as HCl/air/oxygen or CCl₄, may be used, or Deacon's process conditions may be used. The conversion of 244bb to 1234yf may be accomplished using a chlorine gas free radical initiator, the possible mechanism of which is shown in Scheme 5.

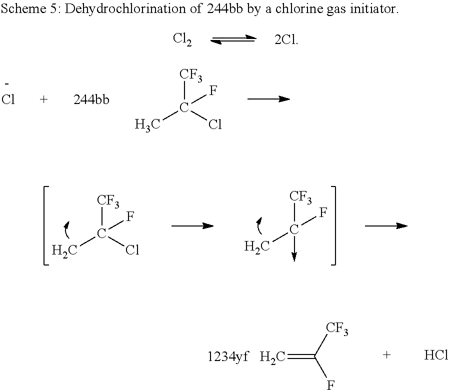

The dehydrochlorination process may be conducted using any suitable conditions. For example, the dehydrochlorination of 244bb using a chlorine gas free radical initiator may be carried out at temperature of about 200-600° C., preferably about 300-500° C. for a contact time of about 1-100 seconds. The percent of chlorine gas may be present in any effective amount, for example, about 0.1-4.0 volume % of 244bb, preferably between 0.5-2 volume %. Other free radical chlorine initiators, such as CCl₄, may be used in effective amounts of about 0.1-4 volume % of 244bb.

Alternatively, or in addition, the intermediate may be dehydrochlorinated in the presence of a transition metal-based catalyst (e.g., a nickel-based catalyst) as the dehydrochlorination catalyst. For example, the dehydrochlorination of 244bb to 1234yf may be accomplished by using a catalytic gas phase dehydrochlorination catalyst, such as a nickel salt-based catalyst, which may be supported or unsupported. Any suitable dehydrochlorination catalyst may be used, such as a catalyst comprising Cu, Co, Cr, Ni, Zn, etc., which may be supported or unsupported. If supported, the support may be selected from alumina, fluorinated alumina, chromia, activated carbon, etc. The catalyst may be of any suitable form, such as anhydrous, powder, pelletized, etc. In an exemplary embodiment, the catalyst is an anhydrous nickel-based catalyst. In another exemplary embodiment, the catalyst is a CuCl₂/alumina catalyst and the dehydrochlorination of 244bb to 1234yf occurs by catalytic oxychlorination. The catalyst may also be activated or re-activated using dry air and anhydrous HCl gas. The mechanism of the HCl elimination may occur as shown in Scheme 6.

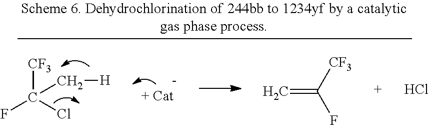

The dehydrochlorination process may be conducted using any suitable conditions. For example, 244bb may be dehydrochlorinated under Deacon's process conditions by co-feeding air over the solid catalyst, e.g., Ni, which may be supported or unsupported. The level of oxygen feed, e.g., as air, may be about 0.1-1 volume %.

Accordingly, in an exemplary embodiment, the converting step is a two-step process comprising:

(a) fluorinating 1,1,1,2-tetrachloro-2-fluoropropane (HCFC-241bb) to form 1,1,1,2-tetrafluoro-2-chloropropane (HCFC-244bb); and

(b) dehydrochlorinating 1,1,1,2-tetrafluoro-2-chloropropane (HCFC-244bb) in the presence of a catalyst, e.g., chlorine gas and/or anhydrous nickel salt, to form 2,3,3,3-tetrafluoropropene (HFO-1234yf).

### Synthesizing 241bb, 244bb, and 1234yf

As discussed above 241bb and intermediate 244bb may be used to produce the tetrafluoroolefin. Another aspect of the present invention includes producing 241bb and/or 244b using routes with high selectivity and little or no corrosion which would be practical to carry out on an industrial scale. In an exemplary embodiment, 1,1,1,2-tetrachloro-2-fluoropropane (HCFC-241bb) is formed by dehydrochlorinating 1,2,3-trichloropropane (HCC-260da) to form 2,3-dichloropropene (HCO-1250xf); fluorinating 2,3-dichloropropene (HCO-1250xf) to form 1,2-dichloro-2-fluoropropane (HCFC-261bb); and chlorinating 1,2-dichloro-2-fluoropropane (HCFC-261bb) to form 1,1,1,2-tetrachloro-2-fluoropropane (HCFC-241bb). The 241bb may be converted to 1234yf using any of the processes described herein. Alternatively, the 241bb may be converted into 244bb, which may be used, for example, in the elimination process discussed above to form 1234yf.

Trichloropropane Feed

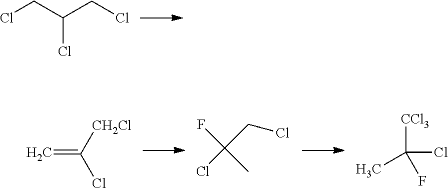

241bb and 244bb may be produced by using trichloropropane (TCP) as a feedstock. TCP has the molecular formula C₃H₅Cl₃. Isomers of trichloropropane include 1,1,1-trichloropropane, 1,1,2-trichloropropane, 1,2,2-trichloropropane, 1,2,3-trichloropropane, and 1,1,3-trichloropropane. In an exemplary embodiment, the trichloropropane is 1,2,3-trichloropropane. 1,2,3-Trichloropropane may be purchased or manufactured, for example, by thermal or photochlorination of allyl chloride.

241bb and the intermediate 244bb may be prepared on a practical industrial route by starting with 1,2,3-trichloropropane (HCC-260da), for example. First, 1,2,3-trichloropropane (HCC-260da) is dehydrochlorinated to produce 1250xf. Liquid phase fluorination of 1250xf may produce 261bb. Upon chlorination, 261bb may produce 241bb. The 241bb maybe used in the processes described herein to produce 1234yf. Alternatively, or in addition, the 241bb may be subjected to liquid phase fluorination, for example, using a mild Lewis acid catalyst, to produce 244bb selectively and without corrosion, as shown in Scheme 7.

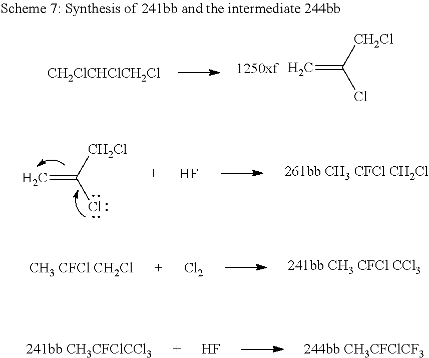

The dehydrochlorination of 1,2,3-trichloropropane (HCC-260da) to 1250xf may be carried out using any suitable method known in the art, e.g., using 40% of sodium hydroxide in an ethanol solution. In a preferred embodiment, dehydrochlorination of 1,2,3-trichloropropane (HCC-260da) to 1250xf is carried out using an aqueous sodium hydroxide solution or catalytically in the gas phase. When the dehydrochlorination occurs catalytically in the gas phase, a supported or unsupported catalyst, such as FeCl₃, may be used. In a preferred embodiment, a FeCl₃ catalyst (e.g., 1-10 weight %) supported on activated carbon is used during the dehydrochlorination. Thus, in step (a), 1,2,3 trichloropropane (HCC-260da) may be dehydrochlorinated using an aqueous sodium hydroxide solution or catalytically in a gas phase using iron chloride supported on activated carbon. Any suitable conditions may be employed. For example, the catalytic dehydrochlorination may occur at a temperature of about 100-400° C., preferably between 200-300° C. at a contact time within the range 1-60 seconds, advantageously between 10-30 seconds. The operating pressure is not particularly critical and may be between 1-20 bar pressure.

1250xf may be converted to 261bb using any suitable method, such as hydrofluorination. In an exemplary embodiment, the hydrofluorination may be carried out continuously in the liquid phase or gas phase. When the hydrofluorination process is carried out in the liquid phase, it is preferred to use a weak Lewis acid selected from TiCl₄, SnCl₄, TaCl₅, etc. Other solid catalysts, such as a Lewis acid comprising a metal selected from titanium, tin, antimony, tantalum, and the like, may also be used. The catalyst may be supported or unsupported. In an exemplary embodiment, the catalyst is supported on a dry, pre-fluorinated activated carbon. Thus, in step (b), 2,3-dichloropropene (HCO-1250xf) may be fluorinated in a liquid phase using a weak Lewis acid. In a preferred embodiment, the catalyst is also subjected to a high pressure HF activation prior to introduction of the 1250xf organic. When the process is carried out continuously in the gas phase, a high surface area supported or unsupported Cr(III) catalyst is preferred. The operating conditions are not particularly limited. The operating temperature may vary between about room temperature to 200° C. The operating pressure is not particularly critical and may be carried out under autogeneous conditions.

261bb may produce 241bb using suitable techniques and conditions known in the art, such as photochlorination in aqueous solution. Conditions have been found, however, which are suitable for selectively photochlorinating under non-aqueous conditions. For example, selective photochlorination under non-aqueous conditions may occur when 261bb is placed in a suitable reactor, such as a quartz tube, with a gaseous chlorine inlet and outlet to allow for the escape of HCl co-product and excess chlorine gas. The quartz tube may then be subjected to UV irradiation. Chlorination may be carried out between 0 to 100° C., preferably between zero and room temperature. The feed rate of chlorine gas and the operating temperature may be adjusted in such a way as to allow for high selectivity, e.g., over 90% of the desired product CH₃CFClCCl₃ (241bb) at very high conversion of the organic feed CH₃CFClCH₂Cl (261bb), preferably above 95%. Thus, in step (c), 1,2-dichloro-2-fluoropropane (261bb) is photochlorinated to 241bb under non-aqueous conditions.

According to another embodiment of the present invention, a method for producing 2,3,3,3-tetrafluoropropene (HFO-1234yf) comprises dehydrochlorinating 1,2,3 trichloropropane (HCC-260da) to form 2,3-dichloropropene (HCO-1250xf); fluorinating 2,3-dichloropropene (HCO-1250xf) to form 1,2-dichloro-2-fluoropropane (HCFC-261bb); chlorinating 1,2-dichloro-2-fluoropropane (HCFC-261bb) to form 1,1,1,2-tetrachloro-2-fluoropropane (HCFC-241bb); and converting 1,1,1,2-tetrachloro-2-fluoropropane (HCFC-241bb) to 2,3,3,3-tetrafluoropropene (HFO-1234yf).

241bb may be converted to the tetrafluoroolefin using any of the processes and conditions described herein. For example, conversion of 241bb to 1234yf may be (1) a one-step process comprising fluorinating 1,1,1,2-tetrachloro-2-fluoropropane (HCFC-241bb) in the presence of a catalyst comprising chromium to form 2,3,3,3-tetrafluoropropene (1234yf); (2) a two-step process comprising fluorinating 1,1,1,2-tetrachloro-2-fluoropropane (HCFC-241bb) to form 1,1,1,2-tetrafluoro-2-chloropropane (HCFC-244bb); and dehydrochlorinating 1,1,1,2-tetrafluoro-2-chloropropane (HCFC-244bb) in the presence of chlorine gas to form 2,3,3,3-tetrafluoropropene (HFO-1234yf); or (3) a two-step process comprising fluorinating 1,1,1,2-tetrachloro-2-fluoropropane (HCFC-241bb) to form 1,1,1,2-tetrafluoro-2-chloropropane (HCFC-244bb); and dehydrochlorinating 1,1,1,2-tetrafluoro-2-chloropropane (HCFC-244bb) in the presence of a catalyst comprising a transition metal, e.g., an anhydrous nickel salt, to form 2,3,3,3-tetrafluoropropene (HFO-1234yf).

Dichloropropene Feed

Alternatively, 241bb and 1234yf may be produced by using CH₂═CCl(CH₂Cl) (1250xf) as the feedstock. In this embodiment, the 1,1,1,2-tetrachloro-2-fluoropropane (HCFC-241bb) is formed by fluorinating 2,3-dichloropropene (HCO-1250xf) to form 1,2-dichloro-2-fluoropropane (HCFC-261bb); and chlorinating 1,2-dichloro-2-fluoropropane (HCFC-261bb) to form 1,1,1,2-tetrachloro-2-fluoropropane (HCFC-241bb). Thus, there are only two process steps to produce 241bb and three to produce 1234yf, as shown in Scheme 8.

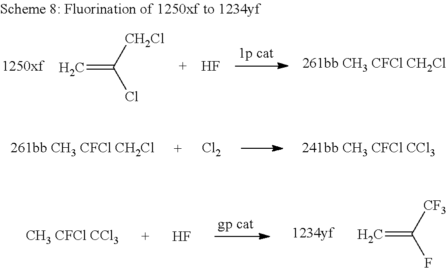

Any suitable fluorination catalysts, co-feeds, and conditions may be used during the fluorination process as would be recognized by one skilled in the art and as described herein. As indicated, 2,3-dichloropropene (HCO-1250xf) may be fluorinated with HF to form 1,2-dichloro-2-fluoropropane (HCFC-261bb) in the presence of a liquid phase (lp) catalyst. 1,2-dichloro-2-fluoropropane (HCFC-261bb) may be chlorinated with chlorine gas to form 1,1,1,2-tetrachloro-2-fluoropropane (HCFC-241bb) in the presence of a gas phase (gp) catalyst.

All of the reactions described herein may be conducted in any suitable reaction vessel or reactor. The vessel or reactor may be of any suitable type, shape, and size. For example, the reactor may be a fixed or fluid catalyst bed reactor, a tubular reactor, etc. The reactions may be carried out batch wise, continuous, or any combination of these. The reactions may be performed using a wide variety of process parameters and process conditions readily ascertainable to one of ordinary skill in the art based on the teachings provided herein. Also, it is known to one of ordinary skill in the art that hydrogen fluoride is corrosive, and the reactors should be constructed accordingly.

The reactions may be carried out in the presence of an inert gas, such as nitrogen, helium or argon. Nitrogen is a preferred inert gas. Also, other gases may be co-fed with the reactants, such as, air, oxygen, or inert gases, such as nitrogen, etc. For example, catalyst activity may be maintained for an extended period of time by co-feeding low levels of oxygen with the tetrachlorofluoropropane during fluorination.

The operating conditions and residence times of the reactants in the reactor should be sufficient for the reactions to take place with an acceptable yield (including conversion efficiency and selectivity), which may be determined as a function of the operating conditions adopted. The reaction pressure can be subatmospheric, atmospheric, or superatmospheric. If a catalyst is used during the reaction and the catalyst deactivates over time, it may be replaced or regenerated using any suitable techniques known in the art.

The hydrofluoroolefin, intermediates, other co-products, and by-products may be formed, such as hydrogen fluoride and hydrogen chloride. Also, some unreacted feed components may be present with the product stream. In certain circumstances, an azeotropic mixture may result. The tetrafluoroolefin, such as HFO-1234yf, may be separated and/or the other intermediates/reactant products or unreacted feedstock may be separated from the tetrafluoroolefin using suitable techniques known to those skilled in the art. For instance, the separation may be accomplished by swing distillation, solvent extraction, membrane separation, scrubbing, adsorption, and the like.

The methods and catalysts described herein produce a tetrafluoroolefin, such as 1234yf, with high selectivity and high conversion. The methods of the present invention provide for improved, simplified production of tetrafluoroolefins. The methods according to the invention exhibit good performance and characteristics especially for the production of the tetrafluoroolefin, 1234yf.

### EXAMPLES

### Prophetic Example 1

### Catalytic Dehydrochlorination of 1,2,3-Trichloropropane to 1250xf

The dehydrochlorination of CH₂ClCHClCH₂Cl (1,2,3-trichloropropane) to CH₂═CCl(CH₂Cl) (1250xf) may be carried out using a fixed bed reactor fitted into an organic gas inlet. The reactor may be heated up electrically using a three-zone furnace. After loading the catalyst (e.g., 20 CC of 5 weight % anhydrous FeCl₃ supported on activated carbon (e.g., CALGON CPG, which is an activated carbon obtainable from Calgon Carbon Corp. with offices in Pittsburgh, Pa.)). Organic feedstock may be fed using a pump at a feed rate, e.g., corresponding to about 20 seconds contact time, and at atmospheric pressure. The organic product may be scrubbed of HCl gas and dried using anhydrous CaSO₄. It is estimated that conversion would be about 12% and selectivity of 1250xf would be about 98%.

### Prophetic Example 2

### Aqueous Dehydrochlorination of 1,2,3-Trichloropropane to 1250xf

1,2,3-trichloropropane (HCC-260da) (e.g., 100 g, 0.678 mole) may be placed in a three necked round bottomed flask, equipped with a 250 ml dropping funnel, water condenser, and mechanical stirrer. Sodium hydroxide aqueous solution (e.g., 115 ml; 0.006 mol/ml) may be added drop wise, with continuous stirring at about 80° C. After complete addition, the reaction mixture may be stirred further at 80° C. for an additional ½ hour. The organic layer may then be separated and dried over anhydrous CaSO₄. The dry organic product may be redistilled to produce about 65 grams (e.g., about 86% yield and 99% purity 1250xf).

### Prophetic Examples 3-5

### Liquid Phase Fluorination of 1250xf to 261bb

The liquid phase fluorination of CH₂═CCl(CH₂Cl) (1250xf)+HF→CH₃CFClCH₂Cl (261bb) may be carried out as follows. A 500 CC autoclave may be fitted with a mechanical stirrer, low temperature condenser, liquid organic inlet, HF gas inlet, catalyst inlet, nitrogen gas inlet, and product outlet. HF (e.g., 200 grams, 10 moles) may be introduced into the autoclave together with the titanium tetrachloride TiCl₄ (e.g., 10 g, 0.053 moles). The mixture may be stirred at room temperature for about ½ hour. The HCl gas may be released and the organic feed 1250xf (e.g., 100 g, 0.9 moles) may be introduced into the reactor. The reaction mixture may be stirred for about 2 hours at 60° C. The HCl gas may be vented. Nitrogen gas (e.g., 40 cm³/m) may be introduced into the reaction mixture. The organic product may be collected in a receiver that has been pre-cooled in a dry ice acetone trap. The product obtained may be about 80 grams, 0.88 moles of CH₃CFClCH₂Cl (261bb) and a small amount of co-product of CH₃CF₂CH₂Cl (262cb). The process may be repeated using SnCl₄ and SbCl₅ as the catalyst. Anticipated results are shown in Table 2.

TABLE 2
Liquid phase fluorination of 1250xf to 261bb

| | | T | % | % | % | % |
|---|---|---|---|---|---|---|
| Example | Catalyst | ° C. | Conversion | 261bb | 262cb | unknown |
| 3 | SbCl₅ | 30 | 100 | 92 | 5 | 3 |
| 4 | TiCl₄ | 30 | 88 | 94 | 6 | 0 |
| 5 | SnCl₄ | 30 | 80 | 96 | 4 | 0 |

### Prophetic Example 6

### Gas Phase Fluorination 1250xf to 261bb using a Solid Sb/C Catalyst

20 cc of Sb/C catalyst (prepared according to U.S. Pat. No. 6,074,985 incorporated herein by reference) may be loaded into a reactor. A mixture of HF gas and organic (e.g., 1.2 molar ratio of 1 HF/1250xf) may be fed together at a feed rate corresponding to about 10 seconds contact time. Excess HF may be scrubbed and dried using anhydrous CaSO₄. It is expected that the % conversion will be about 100 with a selectivity of 96% 261bb and the remainder CH₃CF₂CH₂Cl (262bb).

### Prophetic Example 7

### Photochlorination of 261bb to 241bb

The photochlorination of CH₃CFClCH₂Cl (261bb)+Cl₂→CH₃CFClCCl₃ (241bb) may occur as follows. 1000 ml of 261bb may be placed in a quartz vessel, equipped with a chlorine gas inlet and outlet. A medium pressure Hg ARC may be immersed inside the organic, which may be pre-cooled with water circulation at 5° C. The product may be redistilled at 29° C./2 mm Hg.

### Prophetic Example 8

### Catalyzed Liquid Phase Fluorination of 241bb to 244bb

The liquid phase fluorination of CH₃CFClCCl₃ (241bb)+HF→CH₃CFClCF₃ (244bb) may occur as follows. A catalyst of TiF₄ may be dissolved in HF gas by stirring a mixture TCl₄ (e.g., 10 g, 0.053), and HF (e.g., 200 g, 10 moles) in a 1000 ml autoclave. After releasing all HCl gases, the starting material 1,1,1,2-tetrachloro-2-fluoropropane (241bb) (e.g., 100 g, 0.7 moles) dissolved in 100 ml of 1,1,1,3,3-pentafluorobutane (HFC-365mfc) may be added fast over 10-15 minutes in such a way as to not exceed a certain operating temperature. All HCl gases may be released from the top of the reactor. Intermediate product 244bb may be obtained by venting the product using nitrogen gas 40 cc to a pre-cooled receiver kept at about −78° C.

### Prophetic Example 9

### Dehydrochlorination of 244bb to 1234yf using Chlorine Gas Initiator

The dehydrochlorination of CF₃CFClCH₃ (244bb)→CH₂—CF(CF₃) (1234yf) may occur as follows. A pyrolysis tube may be heated up using a three zone electrical furnace at 500° C., fitted into 244bb and chlorine gas inlets. A mixture of 2.5 volume % 244bb and chlorine gas may be fed in such a way to correspond to about 20 seconds of contact time. HCl co-product and excess chlorine gas may be scrubbed. A 55.6% conversion and 99.4% selectivity to1234yf may result with about a 0.6% selectivity to co-product CH₂═CCl(CF₃) (1233xf).

### Prophetic Example 10

### Dehydrochlorination of 244bb to 1234yf using Activated Carbon and Chlorine Gas Initiator

40 CC of a dry activated carbon may be placed inside a fixed bed reactor. A mixture of chlorine gas and 244bb may be fed over the activated carbon. At 400° C., the conversion is expected to be about 57% with a selectivity of 99.2% to 1234yf.

### Prophetic Example 11

### Dehydrochlorination of 244bb to 1234yf by Catalytic Oxychlorination

A CuCl₂/alumina catalyst may be used inside a fixed bed reactor. A mixture of 244bb and 2 volume % oxygen gas (e.g., introduced as dry air) may be passed over the catalyst bed at a temperature of about 400° C. for 20 seconds contact time. The conversion is expected to be about 55% with a selectivity of 98% to 1234yf.

### Prophetic Example 12

### High Temperature Fluorination of 241bb to 1234yf

The following reaction may occur:

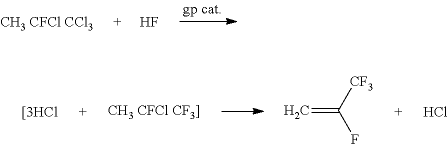

40 cc of a Cr₂O₃ catalyst may be loaded into a fixed bed reactor and activated under pressure using anhydrous HF. After completing the high pressure activation, a mixture of 241bb and HF may be fed over the catalyst bed in a molar ratio of about 5/1, in the presence of 1 volume % oxygen (e.g., as a dry air) and at 200 psig pressure. The organic feed, HF, and air may be adjusted to feed at a contact time corresponding to about 24 seconds. HCl and drying organic may be scrubbed. The selectivity to 1234yf is expected to be about 79%.

### Prophetic Example 13

### Gas Phase Fluorination of 241bb to 1234yf

A catalyst may be prepared using a high pressure activation of Cr₂O₃ according to U.S. Pat. No. 7,485,598, incorporated herein by reference. 20 cc of the high pressure HF activated chrome catalyst may be loaded into Reactor 2 shown in FIG. 1. After catalyst drying for 4 hours at 200° C. using 200 cc of nitrogen gas, a mixture of 100 cc, 4.45 mmol HF and 0.09 gm, 0.45 mmol 241bb, corresponding to a 10 HF/241bb molar ratio, together with 0.5 cc of dry air, may be fed to the reactor. After acid scrubbing and drying organic products, the products may include the following shown in Table 3.

TABLE 3
Catalytic Gas Phase Fluorination of 241bb to 1234yf

| | Example | | | | |
|---|---|---|---|---|---|
| | A | B | C | D | E |
| T ° C. | 200 | 250 | 300 | 350 | 370 |
| Contact time | 48 | 42 | 40 | 36 | 35 |
| seconds | | | | | |
| % conversion | 95 | 100 | 100 | 100 | 100 |
| % 1234yf | 10 | 22 | 55 | 87 | 92 |
| CH₂═CF(CF₃) | | | | | |
| %1233yf | 20 | 18 | 27 | 4 | 4 |
| CH₂═CF(CF₂Cl) | | | | | |
| %1232yf | 55 | 42 | 9 | 3 | 1 |
| CH₂═CF(CFCl₂) | | | | | |
| % 1231yf | 1 | 8 | 5 | 2 | 2 |
| CH₂═CF(CCl₃) | | | | | |
| % 1231ya | 14 | 10 | 4 | 4 | 1 |
| CCl₂═CF(CH₂Cl) | | | | | |

### Prophetic Example 14

### Catalytic Liquid Phase Fluorination of 241bb to 1234yf in the Presence of HF Activated SbCl₅ Catalyst

A 1000 ml MONEL autoclave, equipped with a mechanical stirrer, may be used with a HF gas inlet, organic reactants inlet, and a chlorine gas inlet. SbCl₅ catalyst (10 grams; 0.033 moles) and HF (100 grams; 5 moles) may be added. The mixture may be stirred at room temperature for approximately one hour, to activate the SbCl₅ into SbClₓF_{y} (x+y=5). The produced HCl may be vented from the top of a condenser and maintained at −5° C. using a circulating cooling bath kept at approximately −15° C. After completing catalyst activation, organic 241bb (50 grams, 0.25 moles) may be added to the reaction mixture, which may be heated up to 110° C. with continuous stirring for approximately one hour and approximately 600 psi autogeneous pressure. The reaction mixture may be vented with continuous flow of 40 cc of nitrogen into a water scrubber for about 10 hours. Subsequently, the mixture may be dried using an anhydrous CaSO₄ bed. Volatile organic product may be collected in a cold bath kept at −78° C. using a dry ice acetone mixture. The heavy organic and unreacted product may be analyzed using gas chromatography. The total conversion is estimated at 100% and selectivity of the product obtained (based on 241bb) is estimated as follows: 6% 1234yf; 85% 244bb; 2% 1232yf; 2% 1231yf, 4% 1231ya; and 1% of unidentified products. Similarly, the process may be carried out using different levels of antimony catalyst, as shown in Examples F-I in Table 4.

TABLE 4
Catalytic Liquid Phase Fluorination of 241bb to 1234yf

Examples

| | F | G | H | I |
|---|---|---|---|---|
| Antimony | .033 | .1 | .15 | .2 |
| % conversion | 100 | 100 | 100 | 100 |
| %1234yf | 6 | 10 | 14 | 14 |
| %244bb | 85 | 81 | 77 | 78 |
| %1232yf | 2 | 2 | 2 | 1 |
| %1231yf | 2 | 2 | 2 | 2 |
| %1231ya | 4 | 4 | 4 | 4 |
| % unidentified | 1 | 1 | 1 | 1 |
| products | | | | |

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A method for producing a tetrafluoroolefin comprising contacting 1,1,1,2-tetrachloro-2-fluoropropane (HCFC-241bb) with or without a catalyst under conditions effective to convert the 1,1,1,2-tetrachloro-2-fluoropropane (HCFC-241bb) to the tetrafluoroolefin, optionally, via an intermediate.

2. A method according to claim 1, wherein the tetrafluoroolefin is 2,3,3,3-tetrafluoropropene (HFO-1234yf).

3. A method according to claim 1, wherein the 1,1,1,2-tetrachloro-2-fluoropropane (HCFC-241bb) is converted into the tetrafluoroolefin using a one-step process comprising fluorinating the 1,1,1,2-tetrachloro-2-fluoropropane (HCFC-241bb) to form the tetrafluoroolefin.

4. A method according to claim 3, wherein hydrogen fluoride is the source of fluorine during the fluorination step.

5. A method according to claim 3, wherein the fluorination step occurs in a gas phase or a liquid phase.

6. A method according to claim 3, wherein the fluorination step occurs in the presence of a chromium containing catalyst in a gas phase fluorination.

7. A method according to claim 6, wherein the fluorination step occurs in the presence of a co-catalyst selected from the group consisting of Zn, Ni, Co, Mn, Mg, and mixtures thereof.

8. A method according to claim 3, wherein the fluorination step occurs in the presence of a catalyst comprising a superacid in a liquid phase fluorination in the presence or absence of a solvent.

9. A method according to claim 8, wherein the superacid comprises an element selected from the group consisting of Ti, Sn, Nb, Ta, Sb, B, and mixtures thereof.

10. A method according to claim 8, wherein the catalyst comprises antimony halide.

11. A method according to claim 8, wherein the catalyst is subjected to hydrogen fluoride activation.

12. A method according to claim 8, wherein the fluorination step occurs in the presence of a chlorine gas.

13. A method according to claim 1, wherein the 1,1,1,2-tetrachloro-2-fluoropropane (HCFC-241bb) is converted into the tetrafluoroolefin using a two-step process comprising fluorinating the 1,1,1,2-tetrachloro-2-fluoropropane (HCFC-241bb) to form an intermediate; and subsequently, dehydrochlorinating the intermediate to form the tetrafluoroolefin.

14. A method according to claim 13, wherein the dehydrochlorination step occurs in the presence of a dehydrochlorination catalyst.

15. A method according to claim 14, wherein the intermediate is dehydrochlorinated in the presence of a chlorine gas or chlorine gas free radical initiator as the dehydrochlorination catalyst.

16. A method according to claim 14, wherein the intermediate is dehydrochlorinated in the presence of a transition metal-based catalyst as the dehydrochlorination catalyst.

17. A method according to claim 1, wherein the intermediate is 1,1,1,2-tetrafluoro-2-chloropropane (HCFC-244bb).

18. A method for producing a tetrafluoroolefin comprising contacting 1,1,1,2-tetrafluoro-2-chloropropane (HCFC-244bb) with or without a catalyst under conditions effective to convert the 1,1,1,2-tetrafluoro-2-chloropropane (HCFC-244bb) to the tetrafluoroolefin.

19. A method according to claim 18, wherein the 1,1,1,2-tetrafluoro-2-chloropropane (HCFC-244bb) is dehydrochlorinated in the presence of chlorine gas to form 2,3,3,3-tetrafluoropropene (HFO-1234yf).

20. A method according to claim 18, wherein the 1,1,1,2-tetrafluoro-2-chloropropane (HCFC-244bb) is dehydrochlorinated in the presence of a catalyst comprising anhydrous nickel salt to form 2,3,3,3-tetrafluoropropene (HFO-1234yf).

21. A method for producing 2,3,3,3-tetrafluoropropene (HFO-1234yf) comprising converting 1,1,1,2-tetrachloro-2-fluoropropane (HCFC-241bb) to 2,3,3,3-tetrafluoropropene (HFO-1234yf).

22. A method according to claim 21, wherein the converting step is a one-step process comprising fluorinating 1,1,1,2-tetrachloro-2-fluoropropane (HCFC-241bb) to form 2,3,3,3-tetrafluoropropene (HFO-1234yf).

23. A method according to claim 21, wherein the converting step is a two-step process comprising:
(a) fluorinating 1,1,1,2-tetrachloro-2-fluoropropane (HCFC-241bb) to form 1,1,1,2-tetrafluoro-2-chloropropane (HCFC-244bb); and
(b) dehydrochlorinating 1,1,1,2-tetrafluoro-2-chloropropane (HCFC-244bb) to form 2,3,3,3-tetrafluoropropene (HFO-1234yf).

24. A method according to claim 23, wherein the dehydrochlorination step occurs in the presence of a catalyst comprising chlorine gas.

25. A method according to claim 23, wherein the dehydrochlorination step occurs in the presence of a catalyst comprising an anhydrous nickel salt.

26. A method according to claim 21, wherein the 1,1,1,2-tetrachloro-2-fluoropropane (HCFC-241bb) is formed by fluorinating 2,3-dichloropropene (HCO-1250xf) to form 1,2-dichloro-2-fluoropropane (HCFC-261bb); and chlorinating 1,2-dichloro-2-fluoropropane (HCFC-261bb) to form 1,1,1,2-tetrachloro-2-fluoropropane (HCFC-241bb).

27. A method according to claim 21, wherein the 1,1,1,2-tetrachloro-2-fluoropropane (HCFC-241bb) is formed by dehydrochlorinating 1,2,3-trichloropropane (HCC-260da) to form 2,3-dichloropropene (HCO-1250xf); fluorinating 2,3-dichloropropene (HCO-1250xf) to form 1,2-dichloro-2-fluoropropane (HCFC-261bb); and chlorinating 1,2-dichloro-2-fluoropropane (HCFC-261bb) to form 1,1,1,2-tetrachloro-2-fluoropropane (HCFC-241bb).

28. A method for producing 2,3,3,3-tetrafluoropropene (HFO-1234yf) comprising:
(a) dehydrochlorinating 1,2,3 trichloropropane (HCC-260da) to form 2,3-dichloropropene (HCO-1250xf);
(b) fluorinating 2,3-dichloropropene (HCO-1250xf) to form 1,2-dichloro-2-fluoropropane (HCFC-261bb);
(c) chlorinating 1,2-dichloro-2-fluoropropane (HCFC-261bb) to form 1,1,1,2-tetrachloro-2-fluoropropane (HCFC-241bb); and
(d) converting 1,1,1,2-tetrachloro-2-fluoropropane (HCFC-241bb) to 2,3,3,3-tetrafluoropropene (HFO-1234yf).

29. A method according to claim 28, wherein in step (a), 1,2,3 trichloropropane (HCC-260da) is dehydrochlorinated using an aqueous sodium hydroxide solution or catalytically in a gas phase using iron chloride supported on activated carbon or unsupported anhydrous iron chloride.

30. A method according to claim 28, wherein in step (b), 2,3-dichloropropene (HCO-1250xf) is fluorinated in a liquid phase using a weak Lewis acid.

31. A method according to claim 28, wherein in step (c), 1,2-dichloro-2-fluoropropane (HCFC-261bb) is photochlorinated under non-aqueous conditions.

32. A method according to claim 28, wherein step (d) is a one-step process comprising fluorinating 1,1,1,2-tetrachloro-2-fluoropropane (HCFC-241bb) in the presence of a catalyst comprising chromium to form 2,3,3,3-tetrafluoropropene (1234yf).

33. A method according to claim 28, wherein step(d) is a two-step process comprising:
(d1) fluorinating 1,1,1,2-tetrachloro-2-fluoropropane (HCFC-241bb) to form 1,1,1,2-tetrafluoro-2-chloropropane (HCFC-244bb); and
(d2) dehydrochlorinating 1,1,1,2-tetrafluoro-2-chloropropane (HCFC-244bb) in the presence of chlorine gas to form 2,3,3,3-tetrafluoropropene (HFO-1234yf).

34. A method according to claim 28, wherein step (d) is a two-step process comprising:
(d1) fluorinating 1,1,1,2-tetrachloro-2-fluoropropane (HCFC-241bb) to form 1,1,1,2-tetrafluoro-2-chloropropane (HCFC-244bb); and
(d2) dehydrochlorinating 1,1,1,2-tetrafluoro-2-chloropropane (HCFC-244bb) in the presence of a catalyst comprising anhydrous nickel salt to form 2,3,3,3-tetrafluoropropene (HFO-1234yf).

35. A method of forming an intermediate comprising fluorinating 1,1,1,2-tetrachloro-2-fluoropropane (HCFC-241bb) to form 1,1,1,2-tetrafluoro-2-chloropropane (HCFC-244bb).

36. A dehydrochlorination method comprising contacting 1,1,1,2-tetrafluoro-2-chloropropane (HCFC-244bb) with chlorine or a chlorine generator under free radical initiation conditions.
